(19) United States
(12) Patent Application Publication
(10) Pub. No.: US 2023/0158143 A1
(43) Pub. Date: May 25, 2023

(54) PHARMACEUTICAL COMPOSITIONS COMPRISING ADALIMUMAB

(71) Applicant: SHILPA BIOLOGICALS PRIVATE LIMITED, DHARWAD (IN)

(72) Inventor: RAJYASHRI KARUR RAMAKRISHNAN, DHARWAD (IN)

(73) Assignee: SHILPA MEDICARE LTD, RAICHUR (IN)

(21) Appl. No.: 17/922,976

(22) PCT Filed: May 19, 2021
(86) PCT No.: PCT/IB2021/054291

§ 371 Date: Nov. 3, 2022

(30) Foreign Application Priority Data
May 21, 2020 (IN) 202041021357

(51) Int. Cl. A61K 39/395; A61K 47/26; A61K 47/12; A61K 9/08

(52) U.S. Cl. CPC A61K 39/39591; A61K 47/26; A61K 47/12; A61K 9/08

(57) ABSTRACT

The present relates to stable aqueous pharmaceutical composition of adalimumab comprising (a) buffer system (b) a surfactant and (c) a polyol and the process for the preparation thereof, particularly the suitable aqueous pharmaceutical compositions comprising adalimumab which is free of stabilizers selected from group consisting of sodium chloride and/or amino acids.

### FILED OF THE INVENTION

The present invention relates to aqueous pharmaceutical compositions comprising adalimumab which is suitable for long term storage. More particularly the suitable aqueous pharmaceutical compositions comprising adalimumab which is free of stabilizers selected from group consisting of sodium chloride and/or amino acids. The invention also provides methods of manufacture of compositions, methods of their administration, and kits containing the same.

### BACKGROUND OF THE INVENTION

Adalimumab is a recombinant human IgG1 monoclonal antibody specific for human TNFα (Tumor necrosis factor alpha). Adalimumab consists of 1330 amino acids and has a molecular weight of approximately 148 kilodaltons. Adalimumab has been described and claimed in U.S. Pat. No. 6,090,382. Adalimumab is produced by recombinant DNA technology in a mammalian cell expression system, such as, for example, Chinese Hamster Ovary cells. Adalimumab binds specifically to TNFa and neutralizes the biological function of TNF by blocking its interaction with the p55 cell surface TNF receptors. This mode of action makes Adalimumab suitable for use in treatment of autoimmune diseases such as rheumatoid arthritis, psoriatic arthritis, Crohn's disease, plaque psoriasis, juvenile idiopathic arthritis, ankylosing spondylitis, ulcerative colitis and hidradenitis suppurativa.

Commercially, Adalimumab is available in both single-use prefilled syringe and single-use autoinjector in aqueous form.

The high concentration compositions of adalimumab tend to aggregate during freeze/thaw and in long term storage. The stability of adalimumab can be enhanced either by varying the concentration of the existing excipients or by inclusion of new excipients into the formulation

Sodium Chloride or amino acids are used as a desirable choice to skilled person in order to maintain stability or prevent aggregation of the adalimumab molecule.

U.S. Pat. No. 8,216,583 discloses a liquid aqueous pharmaceutical formulation of adalimumab comprising a buffer selected from group consisting of acetate (sodium acetate), succinate (such as sodium succinate), gluconate, histidine, citrate; tonicity agents which include mannitol and sodium chloride, a detergent including polysorbate 80 and sodium hydroxide for the pH adjustment.

U.S. Pat. No. 9,382,317 discloses a stable aqueous pharmaceutical composition comprising adalimumab, stabilizer consisting essentially of an amino acid selected from glycine, arginine, methionine, or combinations thereof; and sodium chloride or sodium sulfate; wherein the composition is free of buffer and polyol.

U.S. Pat. No. 8,821,865 discloses a formulation comprising 100 mg/mL adalimumab, 1.0 mg/mL of polysorbate 80, 42 mg/mL of mannitol; wherein the formulation has a pH of 4.7 to 5.7 and does not contain a buffer or a salt and wherein injection of the formulation into a human subject results in a Pain Visual Analog Scale (VAS) score of less than 1.0.

WO2018184693A1 discloses the liquid pharmaceutical formulation comprising adalimumab, EDTA, a C2-C4 polyol, an acetate buffer system, sodium chloride and/or arginine, and a non-ionic surfactant wherein the composition has a pH of between 4.8 and 5.4.

U.S. Pat. Nos. 10,093,728; 9,364,542; 9,844,594; 9,821,059; US20150315271; PCT Publications WO2018179138; WO2018169348; WO2018004260; WO2018184692; WO2018162500; WO2018162503; WO2018119142; WO2018124948; WO2018116198; WO2018067987; WO2017122121; WO2016162819; WO2016124588; WO2016120413; WO2016103093; WO2016066688; WO2015177058; WO2015177057; WO2015177059; WO2015151115; WO2015190378; WO2014193821; WO2015090162; WO2013186230; WO2007092772 discloses the pharmaceutical compositions of adalimumab comprising a stabilizer selected from group consisting of sodium chloride and/or amino acids.

In view of the above prior art documents, there is still a need in the art to develop an economic and effective formulation in order to stabilize adalimumab for long term storage. Therefore, herein the present invention provides a formulation free or essentially free of stabilizers selected from group consisting of sodium chloride and/or amino acids.

### SUMMARY OF THE INVENTION

In an embodiment, the invention is related to a stable aqueous pharmaceutical composition comprising adalimumab, a buffer system, a surfactant and a polyol, wherein the said composition is free or essentially free of stabilizers selected from group consisting sodium chloride and/or amino acids.

In certain embodiments the present invention provides a stable aqueous pharmaceutical composition of adalimumab free or essentially free of stabilizers selected from group consisting of sodium chloride and/or amino acids.

In certain embodiments the present invention provides a stable aqueous pharmaceutical composition buffered between pH 4.5 and 6.5.

In yet another embodiment, the invention is related to stable aqueous pharmaceutical composition of adalimumab free or essentially free of stabilizers selected from group consisting of sodium chloride and/or amino acids comprising a buffer system selected from group consisting histidine, succinate, phosphate, citrate, acetate, maleate and tartrate buffers or combination thereof; a surfactant selected from group consisting of polysorbate 80, polysorbate 20, polysorbate 40, sodium dodecyl sulfate and poloxamer, a polyol selected from group consisting of mannitol, xylitol, sorbitol, dextran, propylene glycol and polyethylene glycol, and pH adjusting agents, wherein the composition has a pH of about 4.5 to about 6.5.

In a further embodiment, the present invention provides the stable aqueous pharmaceutical composition comprising adalimumab in a concentration of more than 25 mg/mL and preferably about 25 mg/mL to about 200 mg/mL of adalimumab; a buffer system selected from group consisting histidine, succinate, phosphate, citrate, acetate, maleate and tartrate buffers or combination thereof; a surfactant selected from group consisting of polysorbate 80, polysorbate 20, polysorbate 40, sodium dodecyl sulfate and poloxamer, a polyol selected from group consisting of mannitol, xylitol, sorbitol, dextran, propylene glycol and polyethylene glycol, and pH adjusting agents, wherein the composition has a pH of about 4.5 to about 6.5, and wherein the composition is free of sodium chloride.

In a still further embodiment, the present invention provides the stable aqueous pharmaceutical composition comprising adalimumab in a concentration of more than 25 mg/mL and preferably about 25 mg/mL to about 200 mg/mL of adalimumab; a buffer system selected from group consisting histidine, succinate, phosphate, citrate, acetate, maleate and tartrate buffers or combination thereof; a surfactant selected from group consisting of polysorbate 80, polysorbate 40, polysorbate 20, sodium dodecyl sulfate and poloxamer, a polyol selected from group consisting of mannitol, xylitol, sorbitol, dextran, propylene glycol and polyethylene glycol, and pH adjusting agents, wherein the composition has a pH of about 4.5 to about 6.5, and wherein the composition is free of amino acids.

In yet another embodiment, the present invention provides the stable aqueous pharmaceutical composition consisting of about 25 mg/mL to about 200 mg/mL of adalimumab, succinate buffer, mannitol, polysorbate 80, and pH adjusting agents wherein the composition has a pH of about 4.5 to about 6.5 and wherein the composition is free of sodium chloride.

In a still further embodiment, the present invention provides the stable aqueous pharmaceutical composition consisting of about 25 mg/mL to about 200 mg/mL of adalimumab, succinate buffer, mannitol, polysorbate 80, and pH adjusting agents wherein the composition has a pH of about 4.5 to about 6.5 and wherein the composition is free of amino acids.

In embodiments of the present invention provides a stable aqueous pharmaceutical composition comprising
(a) about 25 mg/mL to about 200 mg/mL adalimumab,
(b) about 5 mg/mL to about 35 mg/mL mannitol,
(c) about 0.1 mg/mL to about 10 mg/mL polysorbate 80 and
(d) a buffer system consisting of succinate,
   wherein the composition has pH of about 4.5 to about 6.5 and wherein the composition is free of stabilizers selected from group consisting of sodium chloride and amino acids.

In further embodiment of the present invention provides a stable aqueous pharmaceutical composition comprising
(a) about 25 mg/mL to about 200 mg/mL adalimumab,
(b) about 5 mg/mL to about 50 mg/mL mannitol,
(c) about 0.1 mg/mL to about 10 mg/mL polysorbate 80 and
(d) a buffer system consisting of citrate,
   wherein the composition has pH of about 4.5 to about 6.5 and wherein the composition is free of stabilizers selected from group consisting of sodium chloride and amino acids.

In a further embodiment, the invention relates to a kit consisting of
(a) about 25 mg/mL to about 200 mg/mL adalimumab,
(b) about 5 mg/mL to about 35 mg/mL mannitol,
(c) 0.1 mg/mL to about 10 mg/mL polysorbate 80 and
(d) a buffer system consisting of succinate,
   wherein the composition has a pH of about 4.5 to about 6.5 and
   wherein the composition is free of sodium chloride.

In another embodiment, the invention relates to a kit consisting of (a) about 25 mg/mL to about 200 mg/mL adalimumab, (b) about 5 mg/mL to about 50 mg/mL mannitol, (c) about 0.1 mg/mL to about 10 mg/mL polysorbate 80 and (d) a buffer system consisting of citrate, wherein the composition has a pH of about 4.5 to about 6.5 and wherein the composition is free of sodium chloride.

In yet another embodiment, the invention is related to the method for treating a disease using the stable aqueous pharmaceutical composition of the present invention wherein the disease is selected from rheumatoid arthritis, psoriatic arthritis, Crohn's disease, plaque psoriasis, juvenile idiopathic arthritis, active ankylosing spondylitis, ulcerative colitis and hidradenitis suppurativa.

In another embodiment the invention is related to a kit or container containing the pharmaceutical composition of the invention.

The details of one or more embodiments of the invention set forth below are illustrative in nature only and not intended to limit to the scope of the invention.

Other features, objects and advantages of the inventions will be apparent from the description and claims.

### DETAILED DESCRIPTION OF THE INVENTION

As used herein the term “without” or “free” means that either no amino acids and/or sodium chloride is present. If reference is made to no amount amino acids and/or sodium chloride, it should be understood as “no detectable amount”. The term “without” or “free” are interchangeable. The amino acids examples are not limited “arginine, glycine, lysine, histidine, glutamic acid, aspartic acid, isoleucine, leucine, alanine, phenylalanine, tyrosine, tryptophan, methionine, serine, proline, cysteine and suitable combination thereof”.

As used herein, the term “buffer system” refers to a buffer that resists change in pH by the action of its acid-base conjugate components. The example of the buffer system includes but not limited to phosphate buffers, citrate buffers, histidine buffers, succinate buffers, acetate buffers, tartrate buffers and combinations thereof

As used herein, the term “long-term storage” or “long term stability” is understood to mean that the pharmaceutical composition can be stored for three months or more, for six months or more, and preferably for one year or more, most preferably a minimum stable shelf life of at least two years. Generally speaking, the terms “long term storage” and “long term stability” further include stable storage durations that are at least comparable to or better than the stable shelf typically required for currently available commercial formulations of adalimumab, without losses in stability that would render the formulation unsuitable for its intended pharmaceutical application. Long-term storage is also understood to mean that the pharmaceutical composition is stored either as a liquid at 25° C±2° C. or 2-8° C. (5° C±3° C.), or is frozen, e.g., at −20° C., or colder. It is also contemplated that the composition can be frozen and thawed more than once.

The term “stable” with respect to long-term storage is understood to mean that adalimumab contained in the pharmaceutical compositions does not lose more than 20%, or more preferably 15%, or even more preferably 10%, and most preferably 5% of its activity of the composition at the beginning of storage.

In an embodiment of the invention, the present invention relates to a stable aqueous pharmaceutical composition comprising
(a) adalimumab,
(b) surfactant,
(c) polyol and
(d) a buffer system
   wherein the composition is free or essentially free of stabilizers selected from group consisting of sodium chloride and/or amino acids.

In embodiments of the present invention, the stable aqueous pharmaceutical composition comprising a buffer system which plays critical role to regulate the stability of formulation. The buffer system is selected from phosphate buffers, citrate buffers, histidine buffers, succinate buffers, acetate buffers, tartrate buffers, tromethamine buffers and the combinations thereof In preferred embodiment the suitable buffer system is a succinate buffer or citrate buffer.

In a most preferred embodiment, the buffer system consists of succinate, and is free of buffer system selected from group consisting of citrate, phosphate, histidine, tartrate, acetate and maleate.

In a further most preferred embodiment, the buffer system consists of citrate and is free of buffer system selected from group consisting of succinate, phosphate, histidine, tartrate, acetate and maleate.

In embodiments of the present invention, the stable aqueous pharmaceutical composition comprises a surfactant selected from group consisting of polysorbate 80, polysorbate 20, polysorbate 40, dodecyl sulfate and poloxamer. In preferred embodiment the surfactant is polysorbate 80.

In embodiments of the present invention, the stable aqueous pharmaceutical composition comprises a polyol selected from group consisting of mannitol, xylitol, sorbitol, dextran, propylene glycol and polyethylene glycol. In preferred embodiment the suitable polyol is mannitol.

In another embodiment, the stable aqueous pharmaceutical composition comprises pH value ranging from 4.5 to about 6.5, more preferably pH of composition is about 5.2.

In another embodiment, the stable aqueous pharmaceutical composition comprises adalimumab generally in a therapeutic amount of up to 200 mg/mL. In a preferred embodiment the therapeutic amount is about 25 mg/mL to about 200 mg/mL. In a more preferred embodiment the therapeutic amount is about 45 mg/mL to about 110 mg/mL.

In another embodiment, the stable aqueous pharmaceutical composition comprises a buffer system generally present in amounts sufficient to formulate adalimumab for therapeutic use at a concentration of about 25 mg/mL to about 200 mg/mL.

In a further embodiment, the stable aqueous pharmaceutical composition comprises surfactant in an amount of about 0.1 mg/mL to about 10 mg/mL.

In another embodiment of the invention the stable aqueous pharmaceutical composition comprises a polyol in an amount of about 5 mg/mL to about 50 mg/mL and more preferably in an amount of about 5 mg/mL to about 35 mg/mL.

In a further embodiment of the invention the stable aqueous pharmaceutical composition shall comprise a pH adjusting agents selected from sodium hydroxide, citric acid, acetic acid and succinic acid.

In yet another embodiment, the invention provides a stable aqueous pharmaceutical composition of adalimumab comprising
(a) about 25 mg/mL to about 200 mg/mL adalimumab,
(b) a buffer system selected from group consisting of histidine, succinate, phosphate, citrate, acetate, maleate and tartrate buffers or combination thereof,
(c) a surfactant selected from group consisting of polysorbate 80, polysorbate 20, polysorbate 40, sodium dodecyl sulfate and poloxamer; and
(d) a polyol selected from group consisting of mannitol, xylitol, sorbitol, dextran, propylene glycol and polyethylene glycol;
   wherein the composition has pH of about 4.5 to about 6.5 and
   wherein the composition is free of sodium chloride.

In yet another embodiment, the invention provides a stable aqueous pharmaceutical composition of adalimumab comprising
(a) about 25 mg/mL to about 200 mg/mL adalimumab,
(b) a buffer system selected from group consisting of histidine, succinate, phosphate, citrate, acetate, maleate and tartrate buffers or combination thereof,
(c) a surfactant selected from group consisting of polysorbate 80, polysorbate 20, polysorbate 40, sodium dodecyl sulfate and poloxamer and
(d) a polyol selected from group consisting of mannitol, xylitol, sorbitol, dextran, propylene glycol and polyethylene glycol,
   wherein the composition has pH of about 4.5 to about 6.5 and
   wherein the composition is free of amino acids.

In embodiments of the present invention provides a stable aqueous pharmaceutical composition comprising
(a) about 25 mg/mL to about 200 mg/mL adalimumab,
(b) about 5 mg/mL to about 35 mg/mL mannitol,
(c) about 0.1 mg/mL to about 10 mg/mL polysorbate 80 and
(d) a buffer system consisting of succinate,
   wherein the composition has pH of about 4.5 to about 6.5 and wherein the composition is free of stabilizers selected from group consisting of sodium chloride and amino acids.

In further embodiment, the present invention provides the stable aqueous composition comprising adalimumab, wherein the composition has osmolality about 180 mOsM/kg to about 400 mOsM/kg.

In embodiments of the present invention provides a stable aqueous pharmaceutical composition comprising
(a) about 25 mg/mL to about 200 mg/mL adalimumab,
(b) about 5 mg/mL to about 35 mg/mL mannitol,
(c) about 0.1 mg/mL to about 10 mg/mL polysorbate 80 and
(d) a buffer system consisting of succinate,
   wherein the composition has pH of about 4.5 to about 6.5,
   wherein the composition has osmolality of about 180 mOsM/kg to about 400 mOsM/kg and
   wherein the composition is free of stabilizers selected from group consisting of sodium chloride and amino acids.

In yet another embodiment, the present invention provides the stable aqueous pharmaceutical composition consisting of
(a) about 25 mg/mL to about 200 mg/mL adalimumab,
(b) about 5 mg/mL to about 35 mg/mL mannitol,
(c) about 0.1 mg/mL to about 10 mg/mL polysorbate 80 and
(d) a buffer system consisting of succinate,
   wherein the composition has a pH of about 4.5 to about 6.5 and
   wherein the composition is free of sodium chloride.

In a still further embodiment, the present invention provides the stable aqueous pharmaceutical composition consisting of
(a) about 25 mg/mL to about 200 mg/mL adalimumab,
(b) about 5 mg/mL to about 35 mg/mL mannitol,
(c) about 0.1 mg/mL to about 10 mg/mL polysorbate 80 and
(d) a buffer system consisting of succinate,
   wherein the composition has a pH of about 4.5 to about 6.5 and
   wherein the composition is free of amino acids.

The pharmaceutical composition is a sterile and stable for long period of time at 2-8° C. In certain embodiment the pharmaceutical composition is a sterile and stable for long period of time at 25° C.

In another embodiment, the invention relates to a kit consisting of
(e) about 25 mg/mL to about 200 mg/mL adalimumab,
(f) about 5 mg/mL to about 35 mg/mL mannitol,
(g) 0.1 mg/mL to about 10 mg/mL polysorbate 80 and
(h) a buffer system consisting of succinate,
   wherein the composition has a pH of about 4.5 to about 6.5 and
   wherein the composition is free of sodium chloride.

In another embodiment, the invention relates to a kit consisting of
(a) about 25 mg/mL to about 200 mg/mL adalimumab,
(b) about 5 mg/mL to about 35 mg/mL mannitol,
(c) about 0.1 mg/mL to about 10 mg/mL polysorbate 80 and
(d) a buffer system consisting of succinate,
   wherein the composition has a pH of about 4.5 to about 6.5 and
   wherein the composition is free of amino acids.

In a further embodiment, the present invention provides the stable aqueous pharmaceutical composition consisting of
(a) about 100 mg/mL adalimumab,
(b) about 35 mg/mL mannitol,
(c) about 1 mg/mL polysorbate 80 and
(d) a buffer system consisting of succinate.

In a further embodiment, the present invention provides the stable aqueous pharmaceutical composition consisting of
(a) about 100 mg/mL adalimumab,
(b) about 35 mg/mL mannitol,
(c) about 1 mg/mL polysorbate 80 and
(d) a buffer system consisting of succinate,
   wherein the composition has a pH of about 4.5 to about 6.5.

In a further embodiment, the present invention provides the stable aqueous pharmaceutical composition consisting of
(a) about 100 mg/mL adalimumab,
(b) about 35 mg/mL mannitol,
(c) about 1 mg/mL polysorbate 80 and
(d) a buffer system consisting of succinate,
   wherein the composition has a pH of about 4.5 to about 6.5,
   wherein the composition has osmolality of about 180 mOsM/kg to about 400 mOsM/kg and
wherein the composition is free of stabilizers selected from group consisting of sodium chloride and amino acids.

In embodiments of the present invention provides a stable aqueous pharmaceutical composition comprising
(e) about 25 mg/mL to about 200 mg/mL adalimumab,
(f) about 5 mg/mL to about 50 mg/mL mannitol,
(g) about 0.1 mg/mL to about 10 mg/mL polysorbate 80 and
(h) a buffer system consisting of citrate,
   wherein the composition has pH of about 4.5 to about 6.5 and wherein the composition is free of stabilizers selected from group consisting of sodium chloride and amino acids.

In embodiments of the present invention provides a stable aqueous pharmaceutical composition comprising
(a) about 25 mg/mL to about 200 mg/mL adalimumab,
(b) about 5 mg/mL to about 50 mg/mL mannitol,
(c) about 0.1 mg/mL to about 10 mg/mL polysorbate 80 and
(d) a buffer system consisting of citrate,
   wherein the composition has pH of about 4.5 to about 6.5,
   wherein the composition has osmolality of about 180 mOsM/kg to about 400 mOsM/kg and
   wherein the composition is free of stabilizers selected from group consisting of sodium chloride and amino acids.

In yet another embodiment, the present invention provides the stable aqueous pharmaceutical composition consisting of
(a) about 25 mg/mL to about 200 mg/mL adalimumab,
(b) about 5 mg/mL to about 50 mg/mL mannitol,
(c) about 0.1 mg/mL to about 10 mg/mL polysorbate 80 and
(d) a buffer system consisting of citrate,
   wherein the composition has a pH of about 4.5 to about 6.5 and
   wherein the composition is free of sodium chloride.

In a still further embodiment, the present invention provides the stable aqueous pharmaceutical composition consisting of
(a) about 25 mg/mL to about 200 mg/mL adalimumab,
(b) about 5 mg/mL to about 50 mg/mL mannitol,
(c) about 0.1 mg/mL to about 10 mg/mL polysorbate 80 and
(d) a buffer system consisting of citrate,
   wherein the composition has a pH of about 4.5 to about 6.5 and
   wherein the composition is free of amino acids.

In another embodiment, the invention relates to a kit consisting of
(e) about 25 mg/mL to about 200 mg/mL adalimumab,
(f) about 5 mg/mL to about 50 mg/mL mannitol,
(g) about 0.1 mg/mL to about 10 mg/mL polysorbate 80 and
(h) a buffer system consisting of citrate,
   wherein the composition has a pH of about 4.5 to about 6.5 and
   wherein the composition is free of sodium chloride.

In another embodiment, the invention relates to a kit consisting of
(a) about 25 mg/mL to about 200 mg/mL adalimumab,
(b) about 5 mg/mL to about 50 mg/mL mannitol,
(c) about 0.1 mg/mL to about 10 mg/mL polysorbate 80 and
(d) a buffer system consisting of citrate,
   wherein the composition has a pH of about 4.5 to about 6.5 and wherein the composition is free of amino acids.

The kit may be a vial, ampoule, syringe, injection pen (e.g. essentially incorporating a syringe; autoinjector), or intravenous bag. Most suitably the drug delivery device is a syringe, suitably an injection pen. Suitably the syringe is a glass syringe. Suitably the syringe comprises a needle, suitably a 29G ¹/₂″ needle.

The following examples are provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### EXAMPLE 1

### Process for Preparation of Stable Pharmaceutical Compositions of Adalimumab

The drug substance of purified adalimumab is obtained and formulated by using various excipients in the present example as disclosed in Table 1.

TABLE 1
Composition of formulations

| Ingredients | Formulation 1 | | Formulation 2 | |
|---|---|---|---|---|
| Adalimumab | 50 | mg/mL | 100 | mg/mL |
| Polysorbate 80 | 0.1-10 | mg/mL | 0.1-10 | mg/mL |
| Mannitol | 5-35 | mg/mL | 5-35 | mg/mL |
| Succinate Buffer | 40-55 | mM | 40-55 | mM |
| pH | 4.5-6.5 | | 4.5-6.5 | |
| Osmolality | 300 ± 30 | mOsm/kg | 300 ± 30 | mOsm/kg |
| Conductivity | 4.5-6.5 | mS/cm | 4.5-6.5 | mS/cm |
| Viscosity | 2 to 5 | cP | 2 to 5 | cP |

### EXAMPLE 2

### Adalimumab Formulation Composition

TABLE 2
Formulation Composition

| Ingredients | Formulation 3 | |
|---|---|---|
| Adalimumab | 100 | mg/mL |
| Polysorbate 80 | 1 | mg/mL |
| Mannitol | 35 | mg/mL |
| Succinate Buffer | 40-55 | mM |
| pH | 5.2 | |
| Osmolality | 300 ± 30 | mOsm/kg |
| Conductivity | 4.5-6.5 | mS/cm |
| Viscosity | 2 to 5 | cP |

### EXAMPLE 3

### Adalimumab Formulation Composition

TABLE 3
Formulation Composition

| Ingredients | Formulation 4 (mg/mL) | |
|---|---|---|
| Adalimumab | 100 | mg |
| Polysorbate 80 | 1 | mg |
| Mannitol | 35 | mg |

| | |
|---|---|
| Succinate Buffer | Q.S to adjust |
| | the pH to 5.2 |

Process for Preparation

Adalimumab concentrate was mixed with required quantity of polysorbate 80 and mannitol. The formulation was adjusted to pH 5.2 with succinate buffer, wherein the concentration of adalimumab in the formulation was 100 mg/mL. Further the adalimumab 100 mg/mL was filled into prefilled syringe containing 0.4 mL of adalimumab 100 mg/mL (Each prefilled syringe contains 40mg of adalimumab).

### EXAMPLE 4

### Stability Studies of Formulation 4 as Disclosed in Example—3.

The formulation 4 as disclosed in Example—4 was stored at 5° C±3° C. and 25° C±2° C/60%±5% RH for 6 months and the tested for colour, clarity, pH, osmolality and concentration of adalimumab. Further the formulation 4 was tested for purity (% of aggregates, % of monomers and % degradation) by SEC HPLC, reducing SDS PAGE and non-reducing PAGE; charge variants (% acidic variants, % lysine 0 variants, and % of basic variants) by WCX HPLC; and activity determined by bioassay. The results of above stored at 5° C±3° C. and 25° C±2° C/60%±5% RH for 6 months are disclosed in Table 4 and Table 5 respectively.

TABLE 4

| | | Formulation | | |
|---|---|---|---|---|
| | | Formulation 4 stored at 5° C. ± 3° C. | | |
| Time points | | 0 M | 3 M | 6 M |
| Colour | | Colourless | Colourless | Colourless |
| Clarity | | Clear | Clear | Clear |
| pH | | 5.3 | 5.1 | 5.3 |
| Osmolality | | 323 | 316 | 325 |
| Concentration (mg/ml) | | 104.1 | 103.3 | 104.5 |
| SEC HPLC | Aggregates % | 0.43 | 0.43 | 0.50 |
| | Monomer % | 99.57 | 99.57 | 99.50 |
| | Degradation % | 0.00 | 0.00 | 0.00 |
| WCX HPLC | Acidic % | 11.61 | 11.70 | 11.65 |
| | Lysine 0% | 63.29 | 63.19 | 63.09 |
| | Basic % | 25.10 | 25.11 | 25.27 |

| | | | |
|---|---|---|---|
| SDS PAGE (Reducing) | Complies | Complies | Complies |
| SDS PAGE (Non-Reducing) | Complies | Complies | Complies |
| Relative Potency (bioassay) | 123% | 97% | 109% |

TABLE 5

| | | Formulation | | | | |
|---|---|---|---|---|---|---|
| | | Formulation 4 stored at 25° C. ± 2° C./60% ± 5% RH | | | | |
| Time points | | 0 M | 1 M | 2 M | 3 M | 6 M |
| Colour | | Colourless | Colourless | Colourless | Colourless | Colourless |
| Clarity | | Clear | Clear | Clear | Clear | Clear |
| pH | | 5.3 | 5.2 | 5.3 | 5.3 | 5.3 |
| Osmolality | | 323 | 314 | 336 | 327 | 327 |
| Concentration (mg/ml) | | 104.1 | 103.9 | 103.8 | 103.6 | 103.2 |
| SEC HPLC | Aggregates % | 0.43 | 0.79 | 0.87 | 1.00 | 1.17 |
| | Monomer % | 99.57 | 98.91 | 98.74 | 98.38 | 97.88 |
| | Degradation % | 0.00 | 0.30 | 0.39 | 0.62 | 0.95 |
| WCX HPLC | Acidic % | 11.61 | 15.32 | 17.84 | 24.26 | 34.83 |
| | Lysine 0% | 63.29 | 59.34 | 55.53 | 50.78 | 44.05 |
| | Basic % | 25.10 | 25.34 | 26.63 | 24.96 | 21.12 |

| | | | | | |
|---|---|---|---|---|---|
| SDS PAGE (Reducing) | Complies | Complies | Complies | Complies | Complies |
| SDS PAGE (Non-Reducing) | Complies | Complies | Complies | Complies | Complies |
| Relative Potency (bioassay) | 123% | 108% | 89% | 88% | 84% |

### EXAMPLE 5

### Adalimumab Formulation Composition

TABLE 6
Formulation Composition

| Ingredients | Formulation 5 (mg/mL) | |
|---|---|---|
| Adalimumab | 100 | mg |
| Polysorbate 80 | 1 | mg |
| Mannitol | 42 | mg |

| | |
|---|---|
| Citrate Buffer | Q.S to adjust |
| | the pH to 5.2 |

Process for Preparation

Adalimumab concentrate was mixed with required quantity of polysorbate 80 and mannitol. The formulation was adjusted to pH 5.2 with citrate buffer, wherein the concentration of adalimumab in the formulation was 100 mg/mL.

Further the adalimumab 100 mg/mL was filled into prefilled syringe containing 0.4 mL of adalimumab 100 mg/mL (Each prefilled syringe contains 40mg of adalimumab).

### EXAMPLE 6

### Stability Studies of Formulation 5 as Disclosed in Example—5

The formulation 5 as disclosed in Example—5 was stored at 5° C±3° C. and 25° C±2° C/60%±5% RH for 6 months and the tested for colour, clarity, pH, osmolality and concentration of adalimumab. Further the formulation 5 was tested for purity (% of aggregates, % of monomers and % degradation) by SEC HPLC, reducing SDS PAGE and non-reducing PAGE; charge variants (% acidic variants, % lysine 0 variants, and % of basic variants) by WCX HPLC and activity by bioassay. The results of above stored at 5° C±3° C. and 25° C±2° C/60%±5% RH for 6 months are disclosed in Table 7 and Table 8 respectively.

TABLE 7

| | | Formulation | | |
|---|---|---|---|---|
| | | Formulation 5 stored at 5° C. ± 3° C. | | |
| Time points | | 0 M | 3 M | 6 M |
| Colour | | Colourless | Colourless | Colourless |
| Clarity | | Clear | Clear | Clear |
| pH | | 5.0 | 5.2 | 5.1 |
| Osmolality | | 286 | 300 | 297 |
| Concentration (mg/ml) | | 103.5 | 102.1 | 102.9 |
| SEC HPLC | Aggregates % | 0.50 | 0.49 | 0.57 |
| | Monomer % | 99.50 | 99.51 | 99.43 |
| | Degradation % | 0.00 | 0.00 | 0.00 |
| WCX HPLC | Acidic % | 11.63 | 11.56 | 11.63 |
| | Lysine 0% | 63.27 | 63.38 | 63.37 |
| | Basic % | 25.10 | 25.06 | 25.00 |

| | | | |
|---|---|---|---|
| SDS PAGE (Reducing) | Complies | Complies | Complies |
| SDS PAGE (Non-Reducing) | Complies | Complies | Complies |
| Relative Potency (bioassay) | 84% | 99% | 93% |

TABLE 8

| | | Formulation | | | | |
|---|---|---|---|---|---|---|
| | | Formulation 5 stored at 25° C. ± 2° C./60% ± 5% RH | | | | |
| Time points | | 0 M | 1 M | 2 M | 3 M | 6 M |
| Colour | | Colourless | Colourless | Colourless | Colourless | Colourless |
| Clarity | | Clear | Clear | Clear | Clear | Clear |
| pH | | 5.0 | 5.1 | 5.1 | 5.1 | 5.2 |
| Osmolality | | 286 | 282 | 290 | 297 | 297 |
| Concentration (mg/ml) | | 103.5 | 103.3 | 104.4 | 103.2 | 103.6 |
| SEC HPLC | Aggregates % | 0.50 | 0.65 | 0.82 | 0.93 | 1.34 |
| | Monomer % | 99.50 | 99.1 | 98.8 | 98.5 | 97.7 |
| | Degradation % | 0.00 | 0.25 | 0.42 | 0.56 | 0.94 |
| WCX HPLC | Acidic % | 11.63 | 14.47 | 17.68 | 23.41 | 35.00 |
| | Lysine 0% | 63.27 | 60.27 | 55.92 | 51.89 | 44.30 |
| | Basic % | 25.10 | 25.26 | 26.40 | 24.70 | 20.80 |

| | | | | | |
|---|---|---|---|---|---|
| SDS PAGE (Reducing) | Complies | Complies | Complies | Complies | Complies |
| SDS PAGE (Non-Reducing) | Complies | Complies | Complies | Complies | Complies |
| Relative Potency (bioassay) | 84% | 113% | 82% | 82% | 110% |

We claim:

1. A stable aqueous pharmaceutical composition comprising
(a) about 25 mg/mL to about 200 mg/mL adalimumab,
(b) about 5 mg/mL to about 35 mg/mL mannitol,
(c) about 0.1 mg/mL to about 10 mg/mL polysorbate 80, and
(d) a buffer system consisting of succinate,
wherein the composition has pH of about 4.5 to about 6.5 and wherein the composition is free of stabilizers selected from group consisting of sodium chloride and amino acids.

2. The composition as claimed in claim 1, wherein the composition has osmolality about 180 mOsM/kg to about 400 mOsM/kg.

3. The composition as claimed in claim 1, wherein pH is about 5.2.

4. The composition as claimed in claim 1, wherein the composition is free of buffer system selected from group consisting of citrate, phosphate, histidine, tartrate, acetate and maleate.

5. A stable aqueous pharmaceutical composition consisting of
(a) about 25 mg/mL to about 200 mg/mL adalimumab,
(b) about 5 mg/mL to about 35 mg/mL mannitol,
(c) about 0.1 mg/mL to about 10 mg/mL polysorbate 80, and
(d) a buffer system consisting of succinate,
wherein the composition has a pH of about 4.5 to about 6.5 and
wherein the composition is free of stabilizers selected from group consisting of sodium chloride and amino acids.

6. A stable aqueous composition as claimed in claim 5 consisting of
(a) about 100 mg/mL adalimumab,
(b) about 35 mg/mL mannitol,
(c) about 1 mg/mL polysorbate 80 and
(d) a buffer system consisting of succinate,
wherein the composition has a pH of about 4.5 to about 6.5.

7. A stable aqueous pharmaceutical composition comprising
(a) about 25 mg/mL to about 200 mg/mL adalimumab,
(b) about 5 mg/mL to about 50 mg/mL mannitol,
(c) about 0.1 mg/mL to about 10 mg/mL polysorbate 80 and
(d) a buffer system consisting of citrate,
wherein the composition has a pH of about 4.5 to about 6.5 and
wherein the composition is free stabilizers selected from group consisting of sodium chloride and amino acids.

8. The composition as claimed in claim 7, wherein the composition has osmolality about 180 mOsM/kg to about 400 mOsM/kg.

9. The composition as claimed in claim 7, wherein pH is about 5.2.

10. The composition as claimed in claim 7, wherein the composition is free of buffer system selected from group consisting of succinate, phosphate, histidine, tartrate, acetate and maleate.
